(19) United States
(12) Patent Application Publication
(10) Pub. No.: US 2025/0041504 A1
(43) Pub. Date: Feb. 6, 2025

(54) DRAINAGE DEVICES, SYSTEMS, AND METHODS

(71) Applicant: BOSTON SCIENTIFIC SCIMED, INC., Maple Grove, MN (US)

(72) Inventors: Louis McNern, Donegal (IE); Stephen McCooey, Dundalk (IE); Michael Walsh, Galway (IE); Deirde Ryan, Tipperary (IE); David Hegarty, Galway (IE); Francis Power, Galway (IE); Hannah Davey, Ennis (IE); Jennifer Anne Smith, Northborough, MA (US); Winston Ronan, Castlerea (IE)

(73) Assignee: BOSTON SCIENTIFIC SCIMED, INC., Maple Grove, MN (US)

(21) Appl. No.: 18/791,756

(22) Filed: Aug. 1, 2024

Related U.S. Application Data
(60) Provisional application No. 63/517,657, filed on Aug. 4, 2023.

(51) Int. Cl. A61M 1/00; A61F 13/05; A61F 13/20

(52) U.S. Cl. CPC A61M 1/916; A61F 13/05; A61M 1/80; A61M 1/84; A61F 2013/2014; A61M 2205/0238

(57) ABSTRACT

Devices, systems, and methods for treating wounds in endoluminal spaces of a patient are disclosed. A device may include an elongate tube defining a proximal end, a distal end, and a lumen, an expandable component adjustably positioned at the distal end of the elongate tube, and a tether coupled to the expandable component and extending proximal from the expandable component through the lumen to a location exterior of the elongate tube. The expandable component may be adjustable between a delivery position within the elongate tube and a fully deployed position at least partially exterior of the elongate tube. The expandable member may be absorbent. A proximal end of the elongate tube may couple to a vacuum source to apply a negative pressure at or proximate the expandable component. An inner member may extend through the elongate tube and be configured to adjustably engage the expandable component.

### CROSS-REFERENCE TO RELATED APPLICATIONS

This application claims the benefit of U.S. Provisional Patent Application Ser. No. 63/517,657, filed Aug. 4, 2023, which is incorporated herein by reference.

### TECHNICAL FIELD

The present disclosure pertains generally, but not by way of limitation, to drainage devices, systems, and methods of treatment. More particularly, the present disclosure relates to a device or system for use in providing vacuum wound therapy to a wound of a patient.

### BACKGROUND

A wide variety of intracorporeal and extracorporeal medical devices and systems have been developed for medical use, for example, for endoscopic procedures. Some of these devices and systems include guidewires, catheters, catheter systems, endoscopic instruments, wound therapy systems, vacuum therapy systems, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices.

In a first example, a device for treating a wound in an endoluminal space of a patient may comprise an elongate tube defining a proximal end, a distal end, and a lumen, an expandable component adjustably positioned at the distal end of the elongate tube between a delivery position within the elongate tube and a fully deployed position at least partially exterior of the elongate tube, wherein the expandable component is absorbent, and a tether coupled to the expandable component and extending proximally from the expandable component through the lumen to a location exterior of the elongate tube.

Additionally or alternatively to any of the examples above, the expandable component may be adjustable to a partially deployed position having a desired volume between a first volume when the expandable component is in the delivery position and a second volume when the expandable component is in the fully deployed position.

Additionally or alternatively to any of the examples above, the elongate tube may be an elongate outer tube and the device may further comprise an elongate inner member configured to extend into the lumen of the elongate outer tube and having a proximal end exterior of the elongate outer tube.

Additionally or alternatively to any of the examples above, the elongate inner member may be configured to engage a proximal portion of the expandable component to adjust a volume of the expandable component deployed from the distal end of the elongate outer tube.

Additionally or alternatively to any of the examples above, the elongate inner member may be an elongate inner tube defining a lumen and the tether may extend through the lumen of the elongate inner tube to a location exterior of the elongate inner tube.

Additionally or alternatively to any of the examples above, a distal end of the tether is coupled to the expandable component at a location of the expandable component distal of the distal end of the elongate tube when the expandable component is in the fully deployed position.

Additionally or alternatively to any of the examples above, the expandable component may be configured to evert as it transitions between the delivery position and the fully deployed position.

Additionally or alternatively to any of the examples above, the expandable component may include a stem portion and a cap portion and the elongate tube may be configured to extend between the stem portion and the cap portion of the expandable component when the expandable component is in the fully deployed position.

Additionally or alternatively to any of the examples above, the device may include a lubricious layer at a location of an outer surface of the expandable component configured to contact the distal end of the elongate tube when the expandable component adjusts between the delivery position and the fully deployed position.

Additionally or alternatively to any of the examples above, the device may include one or more cavities within the expandable component.

Additionally or alternatively to any of the examples above, the expandable component may be a sponge.

Additionally or alternatively to any of the examples above, the expandable component may be configured to be wrung-out when it is positioned within the elongate tube.

In a further example, a system for treating a wound may comprise a vacuum source, an elongate tube defining a lumen, and an expandable component configured to have a first volume associated with a delivery configuration of the expandable component and a second volume associated with a fully deployed configuration of the expandable component, the expandable component is absorbent, and wherein the expandable component may be configured to change volume as the expandable component transitions between the delivery configuration and the fully deployed configuration, and wherein the vacuum source may be in fluid communication with the lumen and the expandable component when the expandable component is in the delivery configuration, the fully deployed configuration, and transitional configurations between the delivery configuration and the fully deployed configuration.

Additionally or alternatively to any of the examples above, the system may further include a tether coupled to the expandable component and extending proximally from the expandable component through the lumen to a location exterior of the elongate tube.

Additionally or alternatively to any of the examples above, the elongate tube may be an elongate outer tube and the system may further includes an elongate inner member configured to extend into the lumen of the elongate outer tube and engage a proximal portion of the expandable component to adjust the expandable component from the delivery configuration to an at least partially deployed configuration.

In a further example, a method of positioning an expandable component at a wound of a patient may comprise inserting the expandable component through a body lumen with the expandable component in a delivery configuration defining a first volume, the expandable component is absorbent, deploying the expandable component from the delivery configuration to a deployed configuration defining a second volume, wherein the second volume is greater than the first volume, applying vacuum pressure to the expandable component when the expandable component is in the deployed configuration, and at least partially re-constraining the expandable component to a configuration at which the expandable component defines a third volume that is less than the second volume while applying the vacuum pressure to the expandable component.

Additionally or alternatively to any of the examples above, the expandable component may be configured to evert when deploying the expandable component from the delivery configuration to the deployed configuration.

Additionally or alternatively to any of the examples above, the method may further include engaging a distal end of an elongate tube with a proximal portion of the expandable component to facilitate the expandable component everting when transitioning from the deployed configuration to the delivery configuration.

Additionally or alternatively to any of the examples above, the method may further include re-deploying the expandable component a re-deployed configuration after re-constraining the expandable component, where the expandable component in the re-deployed configuration defines a fourth volume that is less than the second volume.

Additionally or alternatively to any of the examples above, the method may further include sizing the second volume to be equal to or greater than a volume of the wound at a time the expandable component is deployed from the delivery configuration to the deployed configuration, and sizing the fourth volume to be equal to or greater than the volume of the wound at a time at of re-deploying the expandable component.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a schematic view of an illustrative endoluminal vacuum therapy system;

FIG. 2 illustrates a schematic view of an illustrative device for use in an endoluminal vacuum therapy system;

FIG. 3 is a schematic partial cross-sectional view of an illustrative elongate tube of a device for use in an endoluminal vacuum therapy system;

FIGS. 4A-4E are schematic cross-sectional views of an illustrative technique for deploying and recapturing an expandable component of a device for use in an endoluminal vacuum therapy system;

FIG. 5 is a schematic cross-sectional view of an illustrative expandable component of a device for use in an endoluminal vacuum therapy system;

FIG. 6 is a schematic cross-sectional view of an illustrative expandable component of a device for use in an endoluminal vacuum therapy system;

FIGS. 7A and 7B are schematic side views of an illustrative device for use in an endoluminal vacuum therapy system;

FIGS. 8A-8E are schematic views of an illustrative technique for using an endoluminal vacuum therapy system; and

FIG. 9 is a schematic view of an illustrative technique for using an endoluminal vacuum therapy system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Patients that present with a post-surgical leak or perforation in the gastrointestinal (GI) tract (e.g., after colonic resection, bariatric surgery, esophagectomy, etc.) may have limited treatment options and mortality rates that are high. Existing options for treating post-surgical leaks or perforations include, but are not limited to, endoscopically placed stents, endoscopically placed clips, and re-operating surgically. To reduce migration of implants, prevent walling off infections, increase wound drainage that promotes healing, and reduce morbidity and mortality, additional treatment options are needed.

Negative pressure wound therapy (NPWT) (e.g., vacuum therapy or wound vac) may create an environment around a wound that promotes a healing process. Application of a negative pressure to a wound may assist with removal of wound fluids, which may include infectious materials. A wound vac may be an example of a NPWT configured for external treatment of chronic, non-healing wounds by applying a negative pressure to a wound via a vacuum sealed sponge, where the sponge may be changed at regular intervals over time with increasingly smaller sponges utilized as the wound heals and closes.

Endoluminal vacuum therapy (e.g., EVT or EVAC) may be an adaptation of NPWT that applies NPWT concepts to the GI tract. There is a need for improved EVT or EVAC devices, systems, and methods that treat wounds (e.g., anastomotic leaks, perforations, etc.) in the GI tract and/or other suitable lumens of the human body. The EVT or EVAC devices, systems, and methods disclosed herein may facilitate treating wounds or leaks in the upper and/or lower GI tract, allow for effective placement and replacement of sponges (or alternative material), and/or minimize discomfort to the patient while enabling physicians to have ready-to-use devices and/or systems.

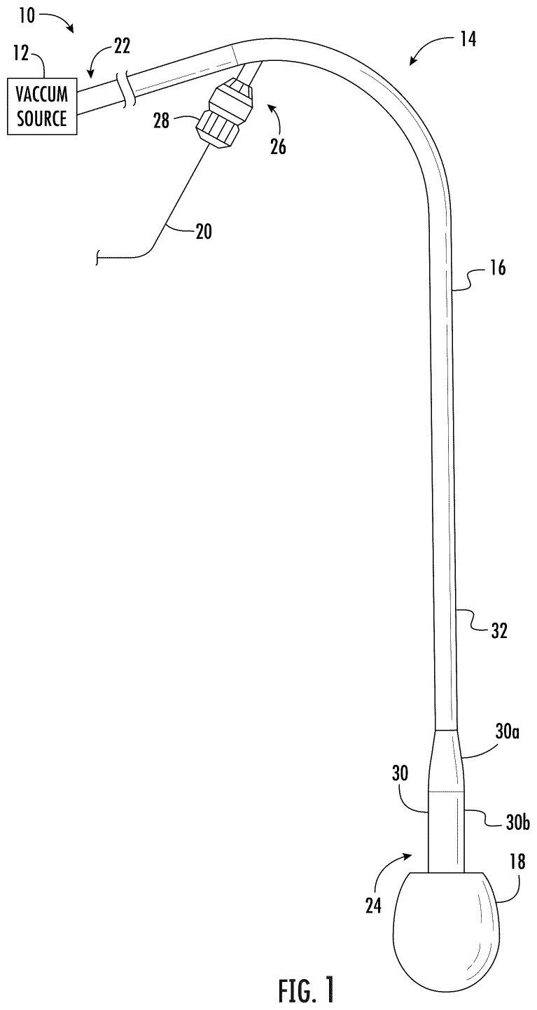

FIG. 1 schematically depicts an illustrative system 10 for treating a wound in an endoluminal space (e.g., a surface, etc.) of a patient. The system 10 may include a vacuum source 12 in fluid communication with a device 14 for insertion into the patient and configured to treat the wound in the endoluminal space (e.g., in an endoluminal surface, etc.).

The vacuum source 12 may be any suitable vacuum source for applying a negative pressure to a wound in an endoluminal surface through the device 14. For example, the vacuum source 12 may be a vacuum source installed in the structure of a building, a vacuum source movable from room to room of a building, and/or other suitable vacuum source couplable to the device 14 to facilitate treating a wound. Although not depicted in FIG. 1, the vacuum source 12 may be in fluid communication with a waste container configured to collect fluid and/or other material from the wound or from locations proximate the wound.

The device 14 configured to facilitate treating a wound in the endoluminal spaces of a patient may include an elongate outer tube 16, an expandable component 18, and a tether 20 configured to extend proximally from the expandable component 18 through a lumen of the outer tube 16 to a location exterior of the outer tube 16. The outer tube 16 may have a proximal end 22 configured to couple with the vacuum source 12 and a distal end 24 configured to be positioned proximate the expandable component 18 in a deployed configuration, as depicted in FIG. 1. The outer tube 16 may be coupled with the vacuum source 12 such that the vacuum source 12 is in fluid communication with a lumen of the outer tube 16 and the expandable component 18 when the expandable component 18 is in the first configuration, a fully deployed or second configuration, and transitional configurations between the first configuration and the fully deployed or second configuration.

The outer tube 16 may have any suitable configuration. In some examples, the outer tube 16 may be a nasogastric (NG) tube, but this is not required and other suitable tube types and/or configurations are contemplated.

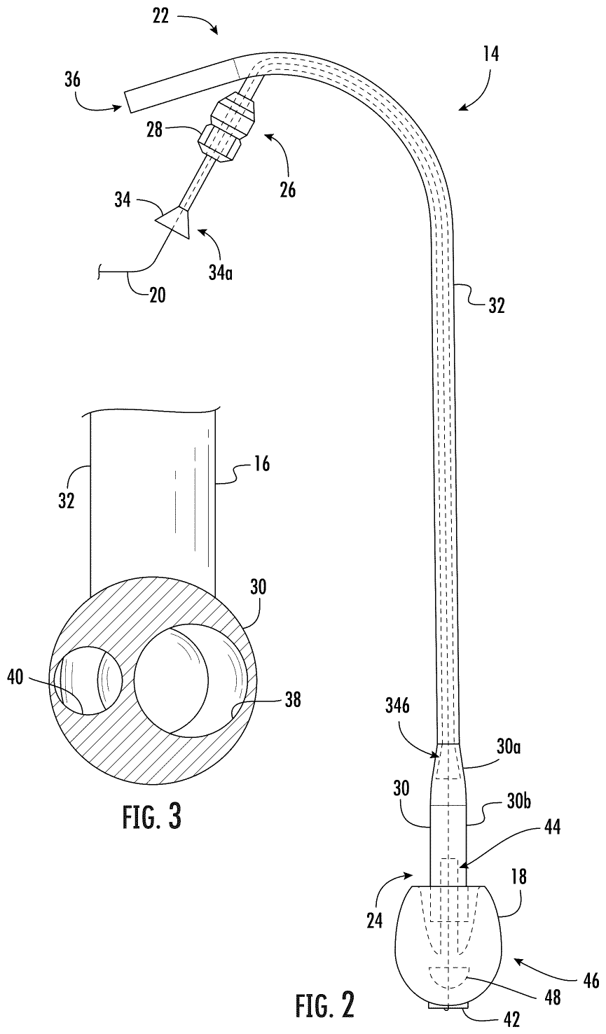

The outer tube 16 may include one or more lumens, where the one or more lumens are configured to facilitate applying a negative pressure from the vacuum source 12 at the expandable component 18 and adjusting the tether 20 to move the expandable component 18 proximate the distal end 24 of the outer tube 16. In some examples, the outer tube 16 may have a single lumen 36, as depicted for example in FIG. 2. In some examples, the outer tube 16 may have two or more lumens.

FIG. 3 schematically depicts a view of a cross-section at an axial location along the containment portion 30 of an illustrative outer tube 16 with a first lumen 38 and a second lumen 40, where the outer tube 16 is bent upward. The first lumen 38 may be larger than the second lumen 40, but this is not required. In some examples, the first lumen 38 may be configured to house the expandable component 18, an inner or pusher member, the tether 20 and/or other suitable components. Additionally or alternatively, the second lumen 40 may be configured to be in communication with the vacuum source 12 and when in communication with the vacuum source 12, the second lumen 40 may facilitate providing a vacuum pressure to the expandable component 18 and/or a target site.

The outer tube 16 may be formed from any suitable biocompatible material configured to navigate body lumens. For example, the outer tube 16 may be formed from a flexible material, a material shaped to be flexible, a material having a desired push-ability for advancing the device 14 through body lumens, a metal material, a polymer material, a combination of metal materials and polymer materials, and/or other suitable materials.

The outer tube 16 may include one or more openings or ports. For example, the outer tube 16 may include a proximal opening or port at or proximate the proximal end 22 configured to couple with the vacuum source 12 and/or other suitable components, a distal opening or port at or proximate the distal end 24 configured to receive the expandable component 18, and an intermediate opening or port 26 (e.g., a side port) between the proximal end 22 and the distal end 24. In some cases, the intermediate opening or port 26 may be configured on the outer tube 16 to be positioned exterior of the patient when the distal end 24 of the outer tube 16 is positioned at a target site.

The lumen(s) of the outer tube 16 may be in fluid communication with one or more of the proximal port, distal port, and the intermediate port 26. In some examples, the expandable component 18 may be retracted through a lumen of the outer tube (e.g., the lumen 36 of a single lumen outer tube 16, the first lumen 38 of a dual lumen outer tube 16, etc.) to allow for the expandable component 18 to be replaced by fully retracting the expandable component 18 through the lumen of the outer tube 16 and out of the intermediate opening or port 26 and then inserting a new expandable component 18 through the intermediate opening or port 26 and through the lumen of the outer tube 16.

The intermediate opening or port 26 may include or be coupled to an adapter 28, but this is not required. When included, the adapter 28 may be configured to selectively or adjustably allow or prevent fluid flow across the intermediate opening or port 26 and/or be configured to secure (e.g., lock) the tether 20 and/or other components relative to the outer tube 16. The adapter 28 may be any suitable type of adapter including, but not limited to, a Touhy-Borst adapter, a pinch valve, a self-sealing valve, and/or other suitable type of adapters or valves.

The adapter 28, when included, may be an integral part of the outer tube 16 and/or may be coupled to the outer tube 16 in any suitable manner. For example, the adapter 28 may be permanently coupled with the intermediate opening or port 26 of the outer tube 16 or adjustably coupled with the intermediate opening or port 26. When adjustably coupled with the intermediate opening or port 26, the adapter 28 may be adjustably coupled in any suitable manner including, but not limited to, via a Luer lock connection and/or other suitable coupling.

The distal end 24 of the outer tube 16 may include a containment portion 30 configured to receive the expandable component 18 in a first configuration (e.g., a delivery or constrained configuration). The containment portion 30 may be elongated and may have an inner diameter configured to receive an entirety of or at least some portion of the expandable component 18.

The containment portion 30 may have a first portion 30a and a second portion 30b, but this is not required. In some examples, the first portion 30a may have a diameter (e.g., an inner and/or outer diameter) tapering proximally to a diameter (e.g., an inner and/or outer diameter) of a core portion 32 of the outer tube 16 and the second portion 30b may extend distally from the first portion 30a and may have a constant or substantially constant inner and/or outer diameter sized to receive the expandable component 18 in the first configuration. In some cases, a distal end of the second portion 30b may have a circumferential flange that distally extends radially outward, which may facilitate re-constraining of the expandable component 18, mitigate tearing of the expandable component during re-constraining, and/or allow for a larger expandable component 18 than if the flange were not included. Other suitable configurations of the containment portion 30 are contemplated.

The tether 20 may be any suitable type of tether coupled to the expandable component 18 and may have a length sufficient to extend exterior of the patient when the device 14 is positioned at or proximate a target site (e.g., a wound, etc.) within the patient. The tether 20 may be and/or may include one or more of a tube, a line, a wire, a string, a shaft, and/or have one or more other suitable configurations. In some cases, the tether 20 may be configured to be flexible, configured to have a tension applied thereto, configured to have sufficient push-ability to adjust the expandable component 18 from the first configuration within the outer tube 16 to an adjusted configuration (e.g., an at least partially expanded or relaxed, deployed configuration or position), where the expandable component 18 is at least partially exterior of the outer tube 16, and/or configured in one or more manners.

FIG. 2 depicts a schematic view of the device 14 utilizing an outer tube 16 with a single lumen 36, where an inner member 34 (e.g., an elongate inner member, a pusher, a deployment and/or re-constraining catheter, and/or other suitable inner member) and the tether 20 extend within the lumen 36 of the outer tube 16. In some cases, the inner member 34 may include one or more lumens through which the tether 20 may extend. The inner member 34 may extend through the intermediate opening or port 26 and be adjustably locked in place relative to the outer tube 16 via the adapter 28.

In some cases, the inner member 34 may be a shaft having a solid cross-section. Additionally or alternatively, the inner member 34 may be tubular and/or otherwise have one or more lumens. When the inner member 34 includes a lumen, the tether 20 may extend through the lumen of the inner member 34 from the expandable component 18 to a location proximal of the proximal end of the inner member 34 and exterior of the patient when the device 14 is positioned with in the patient.

The inner member 34 may have any suitable configuration configured to adjust relative to the outer tube 16 and engage the expandable component 18 to advance and/or withdraw the expandable component 18 relative to the outer tube 16. Although not required, one or more ends of the inner member 34 may flare radially outward. In some examples, a proximal end of the inner member 34 may extend radially outward in the proximal direction to facilitate manipulation of the tether 20 and/or for other purposes. Additionally or alternatively, a distal end of the inner member 34 may extend radially outward in a distal direction to facilitate receiving the tether 20, engaging with the expandable component 18, and/or for other purposes.

A distal end of the tether 20 may be coupled to the expandable component 18. The coupling between the tether 20 and the expandable component 18 may be accomplished in any suitable manner including, but not limited to, via adhesive, a knot, a staple, a rivet, a fusion bond, a melt bond, an anchor, embedding in the expandable component, and/or other suitable coupling mechanism. Further, the tether 20 may be coupled to the expandable component 18 at any suitable location.

In some cases, the tether 20 may engage an anchor 42 at any suitable location along the expandable component 18 configured to facilitate coupling the tether 20 with or relative to the anchor 42. In some examples, the tether 20 may be coupled to the anchor 42 at any location along the expandable component 18 configured to be located distal of a distal end (e.g., a distal terminal end) of the outer tube 16 and/or within an outer diameter of the outer tube 16 when the expandable component 18 is in a deployed configuration to facilitate re-constraining the expandable component 18 within the outer tube 16, as described in greater detail below. In some examples, as depicted in FIG. 2, the tether 20 may be coupled to the anchor 42 at a distal end of the expandable component 18.

The anchor 42 may be or may include any suitable component configured to facilitate fixing the tether 20 relative to the expandable component 18. In some examples, the anchor 42 may be a washer, a stop, a knot, a ball, and/or other suitable component configured to prevent or mitigate pull-through of the tether 20 relative to the expandable component 18.

In some cases, the expandable component 18, the tether 20, and/or the inner member 34 may include one or more indicators at one or more locations to indicate a size (e.g., a diameter, a volume, a length, etc.) of the of the expandable component 18. In some examples, the tether 20 and/or the inner member 34 may include one or more indicators or marks at one or more longitudinal locations configured to extend exterior of the patient that are indicative of a current size of the expandable component 18 exposed to a target site of the patient from the distal end of the outer tube 16 or other component of the device 14. In some examples, the expandable component 18 may include one or more radiopaque markers that may be utilized to determine the current size of the expandable component 18 from an image of the device 14 within the patient.

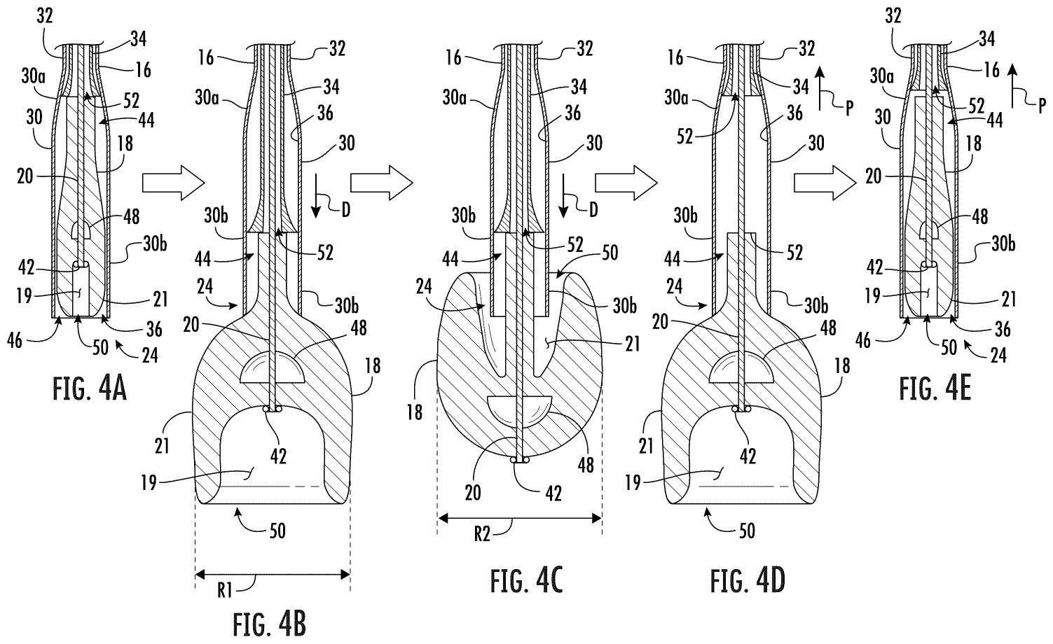

The expandable component 18 may have any suitable shape or configuration configured to expand when deployed from the outer tube 16 and compress when constrained within the outer tube 16 (e.g., within the containment portion 30 of the outer tube 16 and/or other suitable portion of the outer tube 16). As depicted in FIG. 2, the expandable component 18 may have a first portion 44 and a second portion 46, but other suitable configurations are contemplated. The first portion 44 (e.g., a stem portion, etc.) may be elongated and configured to extend within the outer tube 16 when the expandable component 18 is constrained within the outer tube 16 and/or when the expandable component 18 is fully deployed from the outer tube 16. The second portion 46 (e.g., a cap portion) of the expandable component 18 may be configured to have the first configuration when constrained within the outer tube 16 and the adjusted configuration when at least partially deployed from the outer tube 16. In some examples, the second portion 46 of the expandable component 18 is configured to evert when adjusted between the first configuration and a second configuration (e.g., a fully deployed configuration), as discussed in greater detail with respect to FIGS. 4A-4E. Although not required, the first portion 44 and the second portion 46 of the expandable component 18 may form a mushroom-shaped component.

In some cases, the expandable component 18 may include one or more cavities, gaps, or spaces 48 (e.g., empty spaces or spaces filled with less dense or more flexible materials than other portions of the expandable component 18). When included, the one or more gaps or spaces 48 may facilitate constraining and/or re-constraining the expandable component 18 within the containment portion 30 of the outer tube 16 by providing space for material of the expandable component 18 to consume when the expandable component is in or transitioning to the first configuration. Further, in some cases, the one or more gaps or spaces 48 may facilitate defining a shape of the expandable component 18 when in or transitioning to the adjusted configuration or the second configuration as the gaps or spaces 48 may have a preset expandable shape or may be part of a preset expandable shape of the expandable component 18.

The expandable component 18 may be formed from any suitable biocompatible material(s). For example, the expandable component 18 may be formed from a flexible material, an absorbent material (e.g., a material that absorbs liquids, etc.), a polymer material, synthetic absorbable polymers (e.g., poly (lactic-co-glycolic acid), polydioxanone (PDO), etc.), foams, a sponge material, open cell foams, polyurethane foams, hydrogels, alginates, chitosans, cellulose-based materials, silk-based materials, polyvinyl alcohol (PVA) materials, collagen based materials, hyaluronic acid materials, a material capable of being wrung out, a fabric, and/or other suitable biocompatible materials. Additionally or alternatively, the expandable component 18 may be configured from a material that is configured to be contained within the outer tube 16 or other suitable component of the device 14 and self-expand to a predetermined configuration when in the adjusted configuration or the second configuration. In some examples, the expandable component 18 may be and/or may include a sponge and/or other suitable material that may be absorbent and wrung out.

FIGS. 4A-4E depict a schematic cross-sectional view of the expandable component 18 relative to the containment portion 30 of the outer tube 16. The expandable component 18, as depicted in FIGS. 4A-4E, is coupled with the tether 20 via the anchor 42, but this is not required and other coupling configurations are contemplated.

FIG. 4A depicts the expandable component 18 in the first configuration (e.g., the constrained configuration) within the containment portion 30 of the outer tube 16. The expandable component 18 may have a first volume defined by an outer circumference of the expandable component 18 (e.g., defined by at least the outer circumference of the second portion 46 of the expandable component 18).

In the first configuration, the second portion 46 of the expandable component 18 may be fully contained within the containment portion 30 of the outer tube 16, where a distal end of the expandable component 18 (e.g., a first surface 19 at the distal end of the expandable component 18) may define an opening 50 configured to face distally and/or a target site and which may be open to a distal end of the outer tube 16. The opening 50 may be smaller or larger than depicted in FIG. 4A and in some cases, may be entirely or substantially omitted due to first edges or surfaces 19 of the expandable component 18 that define the opening 50 in FIG. 4A approaching each other and in some cases contacting each other. Further, when the expandable component 18 is constrained, the gap or space 48 may be compressed, as depicted in FIG. 4A, such that the surfaces defining the gap or space 48 may approach one another and, in some cases, contact one another. In the first configuration, a second surface 21 may define an exterior surface of the expandable component 18.

The expandable component 18 may be advanced to the target site of the patient while in the first configuration. Once at the target site, the inner member 34 or other suitable component of the device 14 may be advanced distally within the lumen 36 of the outer tube 16 in a direction of arrow D and over the tether 20 that extends through a lumen 52 of the inner member 34, where a distal end of the inner member 34 may engage a proximal end or portion of the expandable component 18 (e.g., a proximal end of the first portion 44 of the expandable component 18) to push the expandable component 18 at last partially out of the containment portion 30 of the outer tube 16 to the adjusted configuration (e.g., a partially constrained or partially deployed and/or expanded configuration), as depicted in FIG. 4B. IN the adjusted configuration, the expandable component may have a first radial width R1 at its widest cross-section (e.g., widest diameter) and an adjusted volume associated with the first radial width R1 that may change as the expandable component 18 is adjusted between the first configuration and the second configuration.

As the expandable component 18 is pushed distally out of the containment portion 30 of the outer tube 16, the second portion 46 of the expandable component 18 self-expands radially outward (e.g., is biased radially outward) or otherwise automatically expands radially relative to the constrained position within the containment portion 30. Alternatively or additionally, the expandable component 18 may be manually expanded using one or more instruments to effect the expansion of the expandable component 18, as desired. As the second portion 46 of the expandable component 18 radially expands, the opening 50, when included, and/or the one or more gaps or spaces 48 may also radially expand outward, as depicted in FIG. 4B relative to FIG. 4A.

FIG. 4C depicts the expandable component 18 pushed fully out of the containment portion 30 of the outer tube 16 by further advancing the inner member 34 distally in the direction of arrow D, such that the expandable component 18 may be in the second configuration (e.g., a fully deployed configuration) with the expandable component 18 in a fully expanded, relaxed position and having a second volume defined by the expandable component 18 (e.g., at least an outer circumference the second portion 44 of the expandable component 18). In some cases, the second volume may be greater than the first volume.

With respect to FIGS. 4B and 4C, the inner member 34 is depicted as being advanced distally to engage the proximal end of the expandable component 18 (e.g., the first portion 44 of the expandable component 18), but this is not required. In some examples, the inner member 34 may be omitted and the tether 20 or other suitable component may be advanced distally in the direction of arrow D to advance the expandable component 18 out of the outer tube 16, where the tether 20 or other suitable component may have a sufficient column strength or buckling load to advance the expandable component 18 from the outer tube 16 without buckling.

When in the second configuration, the expandable component 18 may have a second radial width R2 at its widest cross-section (e.g., widest diameter). The second radial width R2 may be less than, greater than, or equal to the first radial width R1 of the expandable component 18 in a partially deployed or partially constrained position. In some cases, the second volume of the expandable component 18 may be associated with the second radial width R2. When partially deployed, the expandable component 18 may be adjustable to a position having a desired volume between the first volume and the second volume, where the desired volume may be associated with a partially deployed expandable member and the radial width R1.

As depicted in FIG. 4C, the expandable component 18 has everted upon itself relative to the first configuration (e.g., the contained or partially contained configuration) in response to the expandable component 18 being biased to the second configuration and exposed from the outer 16. With the everting of the expandable component 18, the gap or space 48 may evert upon itself relative the configuration of the gap or space 48 depicted in FIG. 4B. When the expandable component 18 is in the second configuration, the anchor 42 may be positioned at the distal end of the expandable component 18, but this is not required and the anchor 42 may be located elsewhere along the expandable component 18 distal of a location along the expandable component 18 at which the distal end of the outer tube 16 initially engages the expandable component 18 between the first portion 44 and the second portion 46 when the expandable component 18 is being re-constrained.

In the second configuration, the opening 50 defined by the expandable component 18 is now facing proximally or toward the outer tube 16. In some cases, the outer tube 16 may be positioned between the first portion 44 and the second portion 46 of the expandable component 18. Further, in the second configuration of the expandable component 18, the second surface 21 is now defining the opening 50 and the first surface 19 may defined an exterior surface of the expandable component 18.

The expandable component 18 may be positioned in or at a wound of a subject when in the second configuration or any at least partially constrained or partially deployed configuration. In some cases, an amount of deployment or constraining of the expandable component 18 may be based on properly sizing the expandable component 18 relative to a wound at which the expandable component 18 may be inserted. Further, the sizing and/or deployment of the expandable component 18 may be performed at or in the wound in which the expandable component 18 is to be placed and/or prior to positioning the expandable component 18 at or in the wound.

FIGS. 4D and 4E schematically depict re-constraining the expandable component 18. The expandable component 18 may be re-constrained for a variety of reasons including, but not limited to, for withdrawal of the expandable component 18 and/or the device 14 from the patient, for resizing the expandable component 18 to better fit a wound over time due to healing of the wound and/or worsening of the wound, for wringing out (e.g., drying out) the absorbent material of the expandable component 18, and/or for other suitable purposes. In some cases, the vacuum source 12 may be active during re-constraining the expandable component 18 such that the negative pressure from the vacuum source 12 pulls any material absorbed by the expandable component 18 and wrung-out by re-constraining the expandable component 18 through the outer tube 16 and out of the device 14.

To initiate re-constraining the expandable component 18, the tether 20 may be withdrawn or advanced in a proximal direction of arrow P. Pulling on the tether 20 may cause the second surface 21 of the expandable component 18 to engage the distal end of the outer tube 16 such that the expandable component 18 everts about the distal end of the outer tube 16 in response to the pulling force on the tether 20 acting on the anchor 42 and the distal end of the expandable component 18 to draw the expandable component 18 into the outer tube 16. When everted in response to pulling on the tether 20, the opening 50 defined by the expandable component 18 may face distally, similar to as depicted in and discussed with respect to FIG. 4B, and the gap or space 48 may evert relative to as shown in FIG. 4C.

When the expandable component 18 is in the second configuration at a wound, the first surface 19 may be engaged with patient tissue at the wound resulting in a shear force acting between the first surface 19 and the patient tissue when the tether 20 and the distal end of the expandable component 18 are initially withdrawn. The shear force, when present, may at least momentarily fix the first surface 19 relative to the patient tissue and facilitate withdrawing a central, distal portion of the expandable component 18 toward the outer tube 16. The withdrawing of the central distal portion of the expandable component 18 toward the outer tube 16 and the distal end of the outer tube 16 engaging the second surface 21 of the expandable component 18 may result in radially inward (e.g., laterally inward) forces pulling the first surface 19 in an axial, central direction (e.g., in the direction of arrows I) of the expandable component 18 and away from lateral walls of the patient tissue to evert the expandable component 18 to a partially constrained configuration, as depicted for example in FIG. 4D.

The configuration of the device 14 resulting in the radially or laterally inward forces may allow the expandable component 18 to remain positioned in or at a wound cavity for an increased amount of time relative to sponge configurations that do not result in a radially or laterally inward force acting on the expandable component 18 because the radially or laterally inward forces are transverse to the shear force and facilitate overcoming the shear force between the expandable component 18 and increased tissue ingrowth at the wound from the expandable component 18 being positioned at the wound for a longer period of time. For example, typical tube and sponge devices are removed every three to five days and a new sponge and tube are inserted during a wound-healing process, whereas the disclosed system 10 or device 14 with the expandable component 18 may be usable for a duration of the wound healing process (e.g., typically longer than three to five days) without being replaced.

Once the expandable component 18 has been everted from the second configuration, as depicted in FIG. 4D, the tether 20 may be further withdrawn in the direction of arrow P and the expandable component 18 may be fully re-constrained within the outer tube 16, as depicted in FIG. 4E, but this is not required and it is contemplated the expandable component 18 may be re-sized and re-deployed without fully re-constraining the expandable component 18. Once the expandable component 18 has been re-constrained and, optionally, wrung out or squeezed out, the expandable component 18 may be re-deployed or withdrawn from the patient. If re-deployed, the expandable component 18 may be reduced in size to account for a decreasing size of a cavity of a wound and may be fully or at least substantially dry due to being wrung out and the vacuum source 12 removing the material from the device 14.

As discussed, the described device 14 and the expandable component 18 may accommodate wounds (e.g., wound cavities, etc.) of varying sizes. Additionally, the device 14 and the expandable component 18 may be utilized with intraluminal cavities, extraluminal cavities, cavities in the stomach, cavities in the duodenum, cavities in the colon, and/or cavities located at one or more other suitable locations. The expandable component 18 may be adjustably sized, shaped, and/or otherwise configured to fill the space of the different cavities (e.g., different anastomotic cavities), as desired.

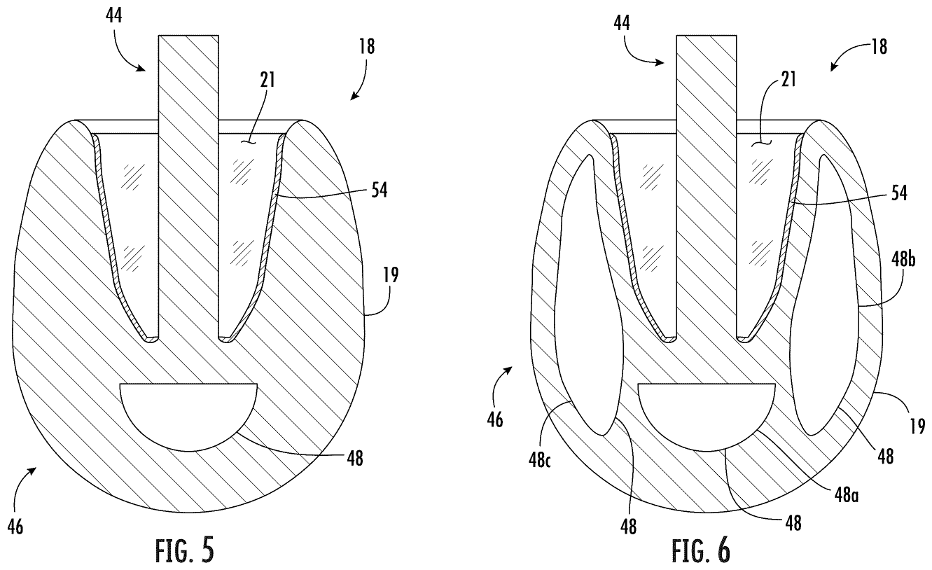

FIG. 5 depicts a schematic cross-sectional view of an illustrative configuration of the expandable component 18 in the second configuration, where the expandable component 18 includes a layer or coating 54 (e.g., a lubricious layer or coating and/or other suitable layer or coating) applied thereto. The coating 54, in some cases, may entirely or at least partially form the second surface 21 defining the opening 50 in the second configuration (e.g., the fully deployed configuration) of the expandable component 18. In some cases, the coating 54 may be applied at additional and/or alternative locations of the expandable component 18 including, but not limited to, at location(s) forming the first surface 19. Further, in addition to or as an alternative to, positioning the coating 54 on the expandable component 18, the coating 54 may be applied to other locations along the device 14 including, but not limited to, along a distal end of the outer tube 16 that interacts with the expandable component 18.

The coating 54 may be any suitable type of coating. The coating 54 may have lubricious properties to reduce friction between the expandable component 18 and the distal end of the outer tube 16, but this is not required. In some examples, the coating 54 may be a flexible material, a lubricious material, a biocompatible material, a polymer material, silicone, polyurethane (PU), polytetrafluoroethylene (PTFE), an elastomer, hydrogels, and/or other suitable type of materials.

FIG. 6 depicts a schematic cross-sectional view of an illustrative configuration of the expandable component 18 in the second configuration, where the expandable component 18 include a plurality of cavities, gaps, or spaces 48. When the expandable component 18 has a circular cross-section, the gaps or spaces 48 may extend entirely or partially circumferentially around a central axis. Although the expandable component 18 is depicted in FIG. 6 as including the coating 54, this is not required and the coating 54 may be omitted or utilized at other suitable locations of the expandable component 18.

In some examples, the second portion 46 of the expandable component 18 may have a first gap or space 48a located between a distal end of the first portion 44 of the expandable component 18 and a distal end of the second portion 46 of the expandable component 18 when the expandable component 18 is in the second configuration, a second gap or space 48b to a first lateral side of the first gap or space 48a, and a third gap or space 48c to a second lateral side of the first gap or space 48a. The gaps or spaces 48 may facilitate compressing or re-constraining the expandable component 18 to facilitate sizing the expandable component 18 for a wound and/or may be configured to facilitate everting the expandable component 18.

The first gap or space 48a may facilitate longitudinal and/or lateral or radial flexibility of the expandable component 18. In some case, an increased flexibility of the distal end of the expandable component due to the first gap or space 48a (e.g., relative to not having a space or gap where the first space or gap 48a is depicted in FIG. 6) facilitates flexing the distal end of the expandable component 18 proximally to initiate everting the expandable component 18 in response to a proximal force applied to the expandable component 18 when the tether 20 (not depicted in FIG. 6) is pulled proximally and a distal end of the outer tube 16 engages the second surface 21.

The second and third gaps or spaces 48b, 48c may be elongated and extend along a side of the first gap or space 48a, but this is not required. In some examples, the second and third gaps or spaces 48b, 48c may facilitate lateral or radially inward flexing of the expandable component 18 to adjust the sizing of the expandable component 18. Further, the configuration of the second and third gaps or spaces 48b, 48c may facilitate everting and re-constraining the expandable component 18.

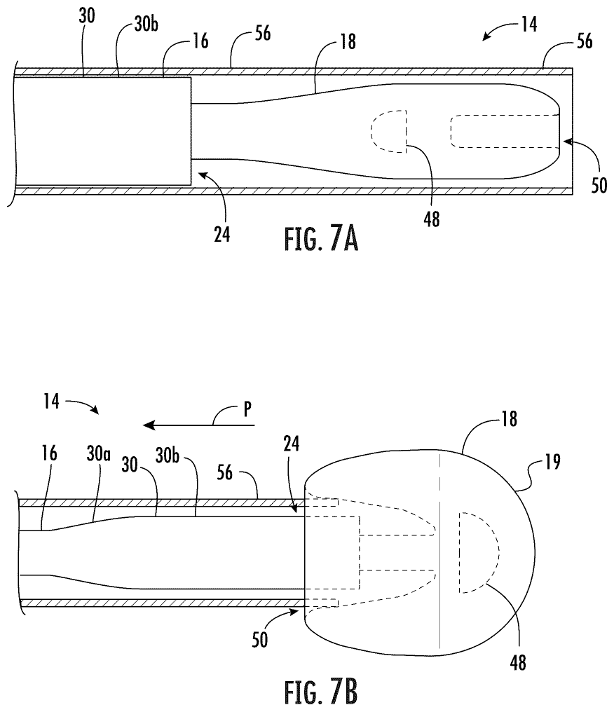

FIGS. 7A and 7B depict a distal portion of an illustrative configuration of the device 14, where the device 14 includes an outer sheath 56 (shown in cross-section) extending over and adjustable with respect to the outer tube 16. In some examples, the outer sheath 56 may be configured to adjust longitudinally about an axis of the outer tube 16 to cover and/or uncover the expandable component 18.

FIG. 7A depicts the device 14 in the first configuration with the outer sheath 56 extending over or otherwise covering the expandable component 18 to maintain the expandable component 18 in a fully constrained or covered position. In some cases, the expandable component 18 may be in the first configuration when delivered to a target site or wound, but this is not required.

When the outer sheath 56 is utilized to maintain the expandable component 18 in the first configuration, the expandable component 18 may be coupled to a distal end of the outer tube 16 in any suitable manner and the tether 20 and/or the inner member 34 may be omitted from the device 14. Alternatively and/or additionally, when the expandable component 18 is coupled to the outer tube 16, the tether 20 and/or other suitable structure may be included with the device 14 to facilitate everting the expandable component 18 when re-constraining the expandable component 18 and/or for other suitable purposes.

In some cases, the expandable component 18 may be coupled with the tether 20 and adjustable relative to the outer tube 16 when the outer sheath 56 is utilized. In such cases, the outer tube 16, the tether 20, and/or the outer sheath 56 may be utilized in combination and/or individually to facilitate everting and re-constraining the expandable component 18.

FIG. 7B depicts the device 14 in the second configuration with the outer sheath withdrawn proximally in the direction of arrow P, which allows the expandable component 18 to evert on itself and fully expand.

As discussed, over time a wound or wound cavity will reduce in size as the wound heals and negative pressure is applied to body tissue at and/or around the wound or wound cavity to remove liquid and/or other material from the wound site. As the wound or wound cavity reduces in size, the expandable component 18 may need to be re-constrained within the outer tube 16 (and/or the outer sheath 56) at regular intervals to prevent or mitigate tissue ingrowth into the expandable component 18.

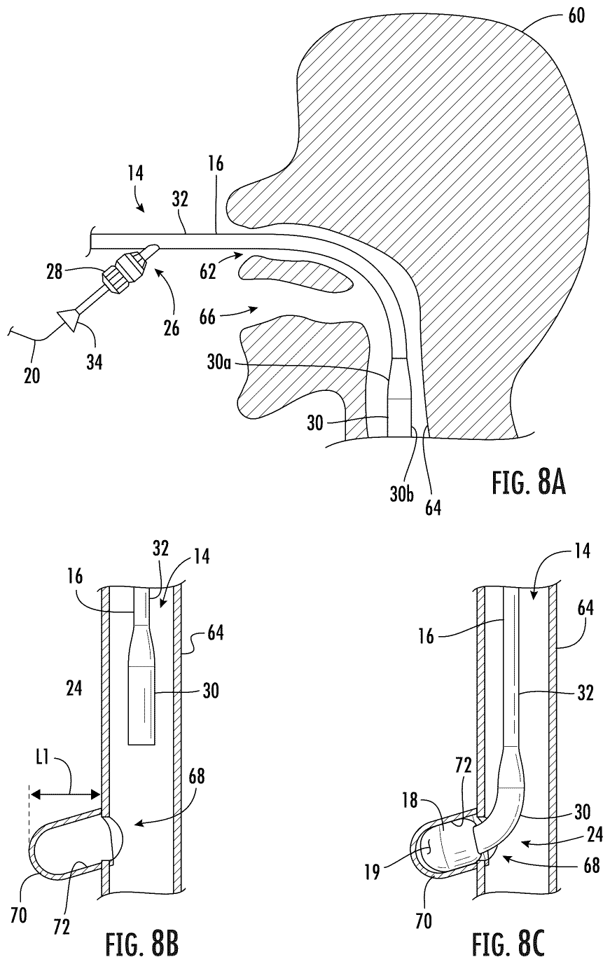

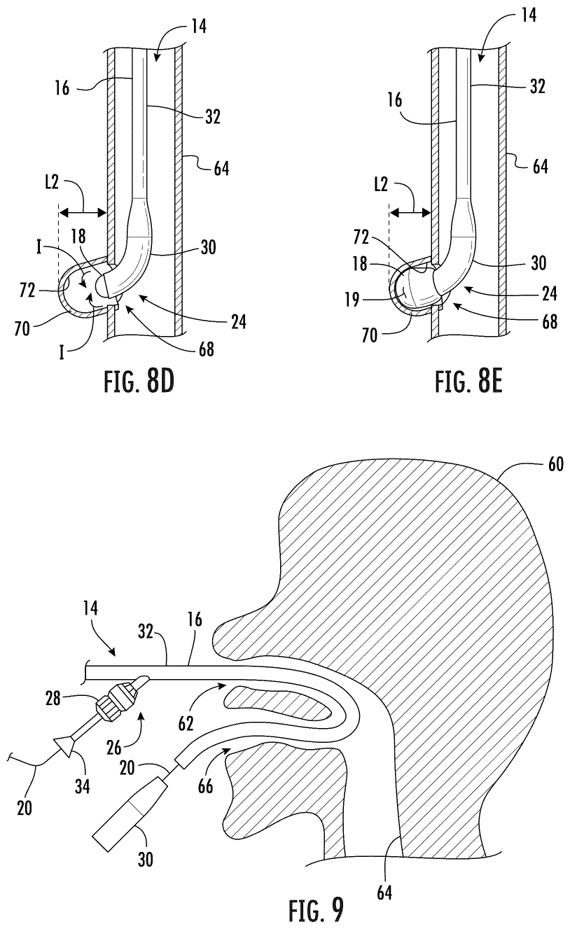

FIGS. 8A-8E depicts schematic steps of an illustrative technique for inserting, positioning, re-constraining, and re-positioning the expandable component 18 at a wound or wound cavity of a patient. Additional or alternative steps for positioning the expandable component 18 at a wound or wound cavity of a subject are contemplated.

FIG. 8A depicts a schematic view of inserting the outer tube 16 of the device 14 through a nasal cavity 62 of a patient 60 and into an esophagus 64 (e.g., a body lumen) of the patient 60. The expandable component 18 (not shown in FIG. 8A) may be in the first configuration defining a first volume within the outer tube 16. Although the device 14 is depicted as being inserted through the nasal cavity 62 of the patient 60, the device 14 may be inserted into the patient 60 through a mouth opening 66 and/or other suitable opening of the patient 60.

FIG. 8B depicts a schematic view of the esophagus 64 with the distal end 24 of the outer tube 16 approaching a target site 68 having a wound cavity 70. As the device 14 is delivered to the target site 68, the expandable component 18 (not depicted in FIG. 8B) may be constrained in the first configuration within the containment portion 30 of the outer tube 16. The wound cavity 70 depicted in FIG. 8B may be defined by inner walls 72 and may have a first lateral length L1 extending from a wall of the esophagus 64.

FIG. 8C depicts a schematic view of the esophagus 64 with the distal end 24 of the outer tube 16 at the target site 68 and the expandable component 18 in the second, fully deployed configuration defining a second volume and positioned in the wound cavity 70. While positioned in the wound cavity 70, a vacuum or negative pressure may be applied to the expandable component to remove liquid and/or other material from proximate the wound cavity 70.

In some cases, the expandable component 18 may be adjusted to a particular size for a size of the wound cavity 70 and then positioned within the expandable component 18. Alternatively, the expandable component 18 may be initially released from the outer tube 16 and expanded while in the wound cavity 70.

Markings and/or indicators on the proximal end of the device 14 may facilitate a user determining a size of the at least partially exposed expandable component 18 within or at the wound cavity 70 to ensure the expandable component 18 is properly sized for the wound cavity 70. When properly sized and positioned in the wound cavity 70, the first surface 19 of the expandable component 18 may engage the interior walls 72 of the wound cavity to facilitate absorbing liquid and/or other material along the wound cavity 70 and such that a shear stress between the first surface 19 and the interior walls 72 may prevent or mitigate the expandable component from backing out of the wound cavity 70.

A size of the expandable component 18 (e.g., a length, a diameter, and/or other suitable size measurement) may be configured to be at the size of the wound cavity 70 or slightly larger so as to create good contact between the expandable component 18 and walls of the wound cavity 70. The good contact between the expandable component 18 and walls of the wound cavity may facilitate the expandable component absorbing, and the negative pressure in the device 14 removing, liquid and/or other material from the wound cavity 70 or the target site 68.

FIG. 8D depicts a schematic view of the esophagus 64 with the expandable component 18 partially re-constrained in the containment portion 30 of the outer tube 16 at which the expandable component 18 has a third volume that is less than the second volume for the purpose of re-sizing the expandable component 18 due to the wound cavity 70 changing size over time (e.g., changing size since the expandable component 18 was initially placed in the wound cavity 70). As depicted in FIG. 8D, a length of the wound cavity 70 has been reduced to a second lateral length L2 extending from the wall of the esophagus 64 from the first lateral length L1 (e.g., as depicted in FIG. 8B) due to healing of the wound cavity 70. The vacuum or negative pressure from the vacuum source 12 may continue to be applied while re-constraining the expandable component 18. In some cases, three to five days, or other suitable amounts of time, may pass between a first placement of the expandable component 18 and a size adjustment of the expandable component 18 to account for changes in sizes of the wound cavity 70.

When re-constraining the expandable component 18, the expandable component 18 may be withdrawn into the outer tube 16 from a central portion of the expandable component 18 such that radially inward forces (e.g., in the direction of arrows I) pull the first surface 19 away from the interior walls 72 of the wound cavity 70 as the expandable component everts. In some cases, the distal end 24 of the outer tube 16 may engage the second surface 21 (not shown in FIG. 8D) to facilitate everting the expandable component 18 and creating the radially inward forces. The radially inward forces assist in overcoming shear forces between the first surface 19 and the interior walls 72 of the wound cavity 70 caused by tissue in-growth and/or other contact between the expandable component 18 and the wound cavity 70.

FIG. 8E depicts a schematic view of the esophagus 64 with the expandable component 18 in a deployed configuration, but with a reduced size (e.g., diameter, volume, etc.) relative to the deployed expandable component in FIG. 8C (e.g., the expandable component 18 may have a fourth volume, where the fourth volume may be less than the second volume depicted in FIG. 4C and equal to or greater than a volume of the wound cavity 70 when the expandable component 18 is re-deployed). To reduce a size of the deployed expandable component 18, a portion of the second portion 46 of the expandable component 18 may remain in or may be withdrawn into the containment portion 30 of the outer tube 16. The vacuum or negative pressure from the vacuum source 12 may continue to be applied to the expandable component 18 when the expandable component 18 is re-deployed.

Although not required, the same expandable component 18 that was deployed in FIG. 8C may be used for treating the wound cavity 70 as the wound cavity 70 adjusts in size over time. Because the expandable component 18 has an adjustable size, the device 14 does not need to be removed from the patient when a different sized expandable component 18 is needed for treating the wound cavity 70.

FIG. 9 depicts a schematic view of inserting the outer tube 16 of the device 14 through a nasal cavity 62 of a patient 60, where the outer tube 16 may have a separable containment portion 30. The outer tube 16 may have a separable containment portion 30 to facilitate utilizing an expandable component 18 that has a larger diameter size in the first configuration (e.g., a delivery configuration) than can comfortably fit through the nasal cavity 62 of the patient. Larger diameter expandable components 18 may be utilized for larger diameter wound cavities and/or for other suitable purposes.

When the outer tube 16 has a replaceable containment portion 30, the tether 20 and the expandable component 18 may be adjustably added to and/or removed from the core portion 32 of the outer tube after the core portion 32 has been passed through the nasal cavity 62 of the patient 60. In operation, the core portion 32 of the outer tube 16 may be inserted through the nasal cavity 62 and out of the mouth opening 66. Once the core portion 32 is extending out of the mouth opening 66, the tether 20 coupled to the expandable component 18 (not shown in FIG. 9) may be threaded through the core portion 32 and the containment portion 30 may be coupled to the core portion 32.

Containment portion 30 may be coupled to the core portion 32 of the outer tube 16 in any suitable manner. In some examples, the containment portion 30 may be coupled to core portion 32 via a snap connection, a ball-detent connection, a threaded connection, a Luer lock connection, and/or other suitable connections.

Once the core portion 32 is coupled with the containment portion 30 and an expandable component 18 is in the first configuration, the outer tube 16 may be partially withdrawn from the patient 60 such that the containment portion 30 of the outer tube 16 is withdrawn into the mouth opening 66 of the patient 60 and aligned with the esophagus 64. Once aligned with the esophagus 64, the outer tube 16 may be further inserted through the nasal cavity until the containment portion 30 and/or the distal end of the outer tube 16 is proximate the target site. Once positioned at the target site, the expandable component 18 may be deployed and/or re-constrained (e.g., by adjusting the inner member 34 through the core portion 32 and the containment portion 30 of the outer tube 16, adjusting the tether 20, and/or adjusting one or more other suitable component of the device 14).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A device for treating a wound in an endoluminal space of a patient, the device comprising:
an elongate tube defining a proximal end, a distal end, and a lumen;
an expandable component adjustably positioned at the distal end of the elongate tube between a delivery position within the elongate tube and a fully deployed position at least partially exterior of the elongate tube, wherein the expandable component is absorbent; and
a tether coupled to the expandable component and extending proximally from the expandable component through the lumen to a location exterior of the elongate tube.

2. The device of claim 1, wherein the expandable component is adjustable to a partially deployed position having a desired volume between a first volume when the expandable component is in the delivery position and a second volume when the expandable component is in the fully deployed position.

3. The device of claim 1, wherein the elongate tube is an elongate outer tube and the device further comprises:
an elongate inner member configured to extend into the lumen of the elongate outer tube and having a proximal end exterior of the elongate outer tube.

4. The device of claim 3, wherein the elongate inner member is configured to engage a proximal portion of the expandable component to adjust a volume of the expandable component deployed from the distal end of the elongate outer tube.

5. The device of claim 3, wherein the elongate inner member is an elongate inner tube defining a lumen and the tether extends through the lumen of the elongate inner tube to a location exterior of the elongate inner tube.

6. The device of claim 1, wherein a distal end of the tether is coupled to the expandable component at a location of the expandable component distal of the distal end of the elongate tube when the expandable component is in the fully deployed position.

7. The device of claim 1, wherein the expandable component is configured to evert as it transitions between the delivery position and the fully deployed position.

8. The device of claim 1, wherein the expandable component includes a stem portion and a cap portion and the elongate tube is configured to extend between the stem portion and the cap portion of the expandable component when the expandable component is in the fully deployed position.

9. The device of claim 1, further comprising:
a lubricious layer at a location of an outer surface of the expandable component configured to contact the distal end of the elongate tube when the expandable component adjusts between the delivery position and the fully deployed position.

10. The device of claim 1, further comprising:
one or more cavities within the expandable component.

11. The device of claim 1, wherein the expandable component is a sponge.

12. The device of claim 1, wherein the expandable component is configured to be wrung-out when it is positioned within the elongate tube.

13. A system for treating a wound, the system comprising:
a vacuum source;
an elongate tube defining a lumen; and
an expandable component configured to have a first volume associated with a delivery configuration of the expandable component and a second volume associated with a fully deployed configuration of the expandable component, the expandable component is absorbent, and
wherein the expandable component is configured to change volume as the expandable component transitions between the delivery configuration and the fully deployed configuration, and
wherein the vacuum source is in fluid communication with the lumen and the expandable component when the expandable component is in the delivery configuration, the fully deployed configuration, and transitional configurations between the delivery configuration and the fully deployed configuration.

14. The system of claim 13, further comprising:
a tether coupled to the expandable component and extending proximally from the expandable component through the lumen to a location exterior of the elongate tube.

15. The system of claim 13, wherein the elongate tube is an elongate outer tube and the system further comprises:
an elongate inner member configured to extend into the lumen of the elongate outer tube and engage a proximal portion of the expandable component to adjust the expandable component from the delivery configuration to an at least partially deployed configuration.

16. A method of positioning an expandable component at a wound of a patient, the method comprising:
inserting the expandable component through a body lumen with the expandable component in a delivery configuration defining a first volume, the expandable component is absorbent;
deploying the expandable component from the delivery configuration to a deployed configuration defining a second volume, wherein the second volume is greater than the first volume;
applying vacuum pressure to the expandable component when the expandable component is in the deployed configuration; and
at least partially re-constraining the expandable component to a configuration at which the expandable component defines a third volume that is less than the second volume while applying the vacuum pressure to the expandable component.

17. The method of claim 16, wherein the expandable component is configured to evert when deploying the expandable component from the delivery configuration to the deployed configuration.

18. The method of claim 16, further comprising:
engaging a distal end of an elongate tube with a proximal portion of the expandable component to facilitate the expandable component everting when transitioning from the deployed configuration to the delivery configuration.

19. The method of claim 16, further comprising:
re-deploying the expandable component a re-deployed configuration after re-constraining the expandable component, where the expandable component in the re-deployed configuration defines a fourth volume that is less than the second volume.

20. The method of claim 19, further comprising:
sizing the second volume to be equal to or greater than a volume of the wound at a time the expandable component is deployed from the delivery configuration to the deployed configuration; and
sizing the fourth volume to be equal to or greater than the volume of the wound at a time at of re-deploying the expandable component.
